(19) United States
(12) Patent Application Publication
(10) Pub. No.: US 2009/0254065 A1
(43) Pub. Date: Oct. 8, 2009

(54) Simple method of transplanting injectable chordrocyte for autologous chondrocyte transplantation

(76) Inventors: Chang-Kwon Ko, Seoul (KR); Eun-Young Lee, Seoul (KR); Jeong-Yong Choi, Gyeonggi-Do (KR); Jae-Deog Jang, Seoul (KR); Cheong-Ho Chang, Seoul (KR); Pyoung-Min Kim, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 11/988,924

(22) PCT Filed: Sep. 23, 2005
(86) PCT No.: PCT/KR2005/003147

§ 371 Date: Jan. 17, 2008

(30) Foreign Application Priority Data
Jul. 20, 2005 (KR) 10-2005-0066026

(51) Int. Cl. A61M 37/00

(52) U.S. Cl. 604/522

(57) ABSTRACT

A method of transplanting an injectable chondrocyte is developed for autologous chondrocyte transplantation, comprising mixing matrices including fibrin, hyaluronic acid and collagen, which are the main ingredients of animal cartilage, and injecting the resulting mixture into a damaged cartilage region. One 1 ml syringe is prepared by mixing 1 ml of a chondrocyte culture with white or light-yellow lyophilized powder (fibrinogen). A second 1 ml syringe is prepared by mixing 1 ml of chondrocyte culture with white lyophilized powder (thrombin), adding 0.1 cc of that mixture to the entire contents of two vials containing chondrocyte suspension, and mixing well. The two syringes are then mounted on a syringe stand, and a mixing tip is mounted to their tips. The contents are mixed in the mixing tip while injecting the resulting mixture into a damaged cartilage region of an animal.

### BACKGROUND OF THE INVENTION

1. Field of the Invention

The present invention relates to a method for transplanting a cartilage therapeutic agent (Chondron: in vitro cultured chondrocytes for autologous chondrocyte transplantation) via injection. More specifically, a method is presented for transplanting a cartilage therapeutic agent involving mixing and transplanting matrices including fibrin, hyaluronic acid and collagen, which are the main ingredients of cartilage, by which the inconvenience of conventional osteoperiosteal grafting is solved, treatment of a wide range of defects and severe osteoarthritis is possible, and the burden on patients during treatment is alleviated via a simplified surgical operation method, thus more rapidly and effectively promoting generation of cartilage and improving consumer satisfaction.

2. Description of the Related Art

Recently, molecular biology, cell biology, developmental biology and tissue engineering, as well known, have contributed to improvements in health and quality of life via development of methods of improving, maintaining, and restoring function to damaged tissues and organs.

Tissue engineering produces tissue substitutes by controlling cell adhesion, growth and differentiation, and activity of cytokines and growth factors via interaction with a cell-based matrix as a tissue replacement to thereby secrete intracellular signaling substances. Early work in tissue engineering has primarily focused on utilization of cells alone or cell therapy utilizing a matrix complex produced by cells, thus greatly limiting applications thereof and leading to an urgent need for the development of more advanced tissue substitutes, and therefore recent research has focused on the development of cartilage therapies utilizing chondrocytes and matrix mixtures.

It has been demonstrated that instability of joints and partial deficiency of menisci can accelerate abrasion and damage to particular surfaces. In addition, particular cartilage, once damaged, cannot be fully regenerated in vivo. Chronic stimulation applied to the damaged particular cartilage causes particular pain and progressive limitation of particular movement, resulting in chronic degenerative arthritis. Chondrocytes that are non-proliferable in vivo can be effectively used in regeneration of joints by inducing differentiation and proliferation of chondrocytes via adjustment of culture conditions in vitro. As a result, transplanted chondrocytes acquire histological and mechanical properties similar to those of cartilage of normal joints, and thus transplantation rejection or tissue destruction is not observed with good results in more than 90% of patients.

Meanwhile, autologous chondrocyte transplantation (ACT) is an effective therapy for treatment of damaged particular cartilage. Other therapies such as abrasion arthroplasty, drilling and microfracture, and autograft of periosteum and perichondrium involve replacement of damaged particular cartilage with fibrocartilage or fibrocartilaginous tissues with no substantial production of hyaline cartilage. However, as compared to any other conventional technique, the most important advantage of ACT lies in the production of hyaline cartilage.

In spite of such an excellent advantage, autologous chondrocyte transplantation (ACT) suffers from some technical and theoretical difficulties as shown in FIG. 1.

That is, firstly, this autologous chondrocyte transplantation technique requires a large incision at the surgical site for collection of periosteum. Such a large incision results in severe pain following the surgery and reduced knee motility.

Secondly, suturing of the periosteal patch on the boundary of a cartilage defect area requires a very complicated surgical technique which has been reported to sometimes be associated with periosteal hypertrophy and intra-particular adhesion. Suturing of periosteum is the most time-consuming process among surgical processes for conducting autologous chondrocyte transplantation.

Additionally, autologous chondrocyte transplantation is disadvantageous in that leakage of chondrocytes away from the transplanted sites sometimes occurs during the rehabilitation process such as therapeutic exercise of the knees after the surgical operation, and the transplanted chondrocytes, which are present in a liquid phase in the transplantation sites, are localized toward one side by the action of gravity, thus resulting in uneven distribution and heterogeneous growth of cartilage.

In order to solve the above-mentioned problems, a great deal of research has focused on the utilization of biodegradable and biocompatible polymers as a carrier or scaffold. For example, a great deal of research into tissue engineering for cartilage in vivo and in vitro is ongoing, utilizing naturally-occurring polymers such as collagen, hyaluronic acid and fibrin as biomaterials, or utilizing synthetic polymers such as polylactic acid or polyglycolic acid.

The scaffold plays an important role in maintenance of a pore structure necessary for growth and proliferation of cells and phenotypes of chondrocytes. Fibrin and hyaluronic acid are suitable polymers satisfying the requirements for use as the scaffold, as mentioned above. Fibrin is a naturally-occurring substance and is a very suitable biological carrier for chondrocyte transplantation. In addition, fibrin has already been reported to have advantages such as biocompatibility, biodegradability and the capability to bind to subchondral bones (bones that are structurally adjacent to, that is, directly beneath the cartilage). Furthermore, fibrin structures can be easily modified into desired shapes and are readily used for injection. Meanwhile, hyaluronic acid is a lubricating agent, found in the synovial joint fluid, and serves to control the moisture content of joints.

However, one problem still remains. Since most scaffolds for tissue engineering are solid types such as patches, sponges and sheets, cartilage therapeutic agents for tissue engineering are difficult to bind to adjacent native cartilage. This fact is of importance in that cartilage therapeutic agents transplanted into cartilage defect regions exert incomplete cure of the boundary region there between.

### SUMMARY OF THE INVENTION

The present invention has been made in view of the above problems, and provides a method for transplantation of a cartilage therapeutic agent via injection.

Therefore, it is a first object of the present invention to solve the inconvenience of conventional osteoperiosteal grafting by mixing and transplanting matrices including collagen, hyaluronic acid and fibrin, which are the main ingredients of cartilage.

A second object of the present invention is to provide convenient in situ transplantation via utilization of rapid gelling characteristics in which an injectable cartilage therapeutic agent is gelated within a short period of time, i.e., about 1 to 5 minutes, upon transplanting.

A third object of the present invention is to enable transplanted sites to be completely bound to the adjacent native cartilage, as well as to enable treatment of a wide variety of cartilage defects and severe osteoarthritis.

A fourth object of the present invention is to alleviate the burden on patients during treatment via a simplified surgical operation method.

A fifth object of the present invention is to more rapidly and effectively promote generation of cartilage, thereby enhancing customer satisfaction via such a simplified surgical operation method.

In accordance with an aspect of the present invention, the above and other objects can be accomplished by the provision of a method of transplanting an injectable chondrocyte for autologous chondrocyte transplantation, the method comprising mixing matrices including collagen, hyaluronic acid and fibrin, which constitute the main ingredients of animal cartilage, via the use of a syringe with a mixing tip, while injecting the resulting mixture into a damaged cartilage region.

In accordance with another aspect of the present invention, there is provided a method of transplanting an injectable chondrocyte for autologous chondrocyte transplantation, comprising;

placing 1 ml of a chondrocyte culture contained in Vial No. 1 (Red) into a 1 ml sterile syringe and injecting the cell culture into Vial No. 2 (Blue) containing white or light-yellow lyophilized powder (fibrinogen) by inserting the syringe into Vial No. 2 in the vertical direction;

injecting 1 ml of the cell culture contained in Vial No. 1 (Red) into Vial No. 3 (Red, small) containing white lyophilized powder (thrombin) by inserting the syringe into Vial No. 3 in the vertical direction, thereby gradually injecting red liquid;

collecting 0.1 cc of the contents of Vial No. 3 (Red, small) using a 1 ml syringe, followed by injection into Vial No. 4 (Yellow);

adding the entire contents of two vials containing chondrocyte suspension to Vial No. 4 (Yellow) using a 1 ml sterile syringe, followed by mixing the contents two or three times using the syringe;

confirming complete dissolution of the contents of Vial No. 2 (Blue) and then introducing the entirety of the dissolved material into a 1 ml syringe;

introducing the well-mixed cell therapeutic of Vial 4 (Yellow) into a 1 ml syringe; and

mounting two 1 ml syringes filled with the contents of Vial No. 2 (Blue) and Vial No. 4 (Yellow) on a standing holder followed by mounting a screw tip on the syringes, and mixing the contents of the syringes using the mixing tip thereof while injecting the resulting mixture into a damaged cartilage region of an animal.

### BRIEF DESCRIPTION OF THE DRAWINGS

FIG. 1 is a photograph showing a conventional method of transplanting a cartilage therapeutic agent.

FIG. 2 is a photograph showing a method of transplanting an injectable cartilage therapeutic agent, which is applied to the present invention.

FIG. 3 is a photograph showing a method of transplanting a cartilage therapeutic agent, which is applied to the present invention.

FIG. 4 is a photograph showing a method of transplanting an injectable cartilage therapeutic agent, which is applied to the present invention, via use of a syringe.

FIG. 5 is a photograph showing physical properties of an injectable cartilage therapeutic agent in accordance with the present invention.

FIG. 6 is a photograph taken 3 months after regeneration of canine cartilage in accordance with an example of the present invention.

FIG. 7 is an EM taken 8 weeks after transplantation of a cartilage therapeutic agent in accordance with the present invention.

FIG. 8 is an EM taken 12 weeks after transplantation of a cartilage therapeutic agent in accordance with the present invention.

FIG. 9 is a photograph taken 12 weeks after regeneration of a canine cartilage tissue in accordance with an example of the present invention;

FIG. 10 is a photograph showing the results of histological examination of a cartilage therapeutic agent in accordance with the present invention.

FIG. 11 is a photograph showing the results of immunohistological examination of a cartilage therapeutic agent in accordance with the present invention.

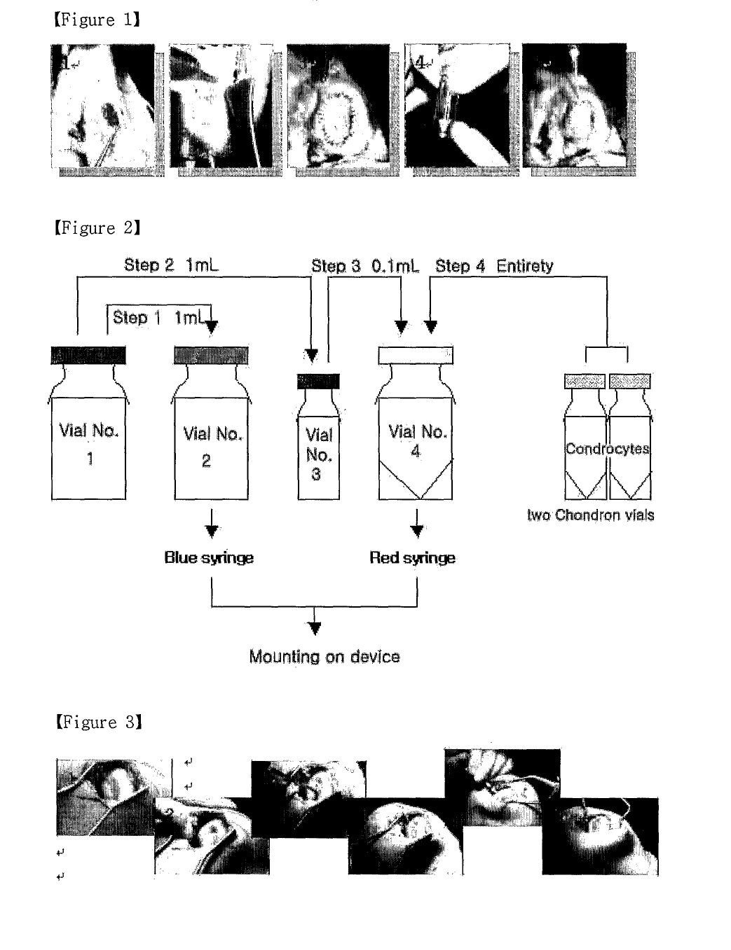

### DESCRIPTION OF THE PREFERRED EMBODIMENTS

The preferred embodiments of the present invention will now be described in more detail with reference to the accompanying drawings.

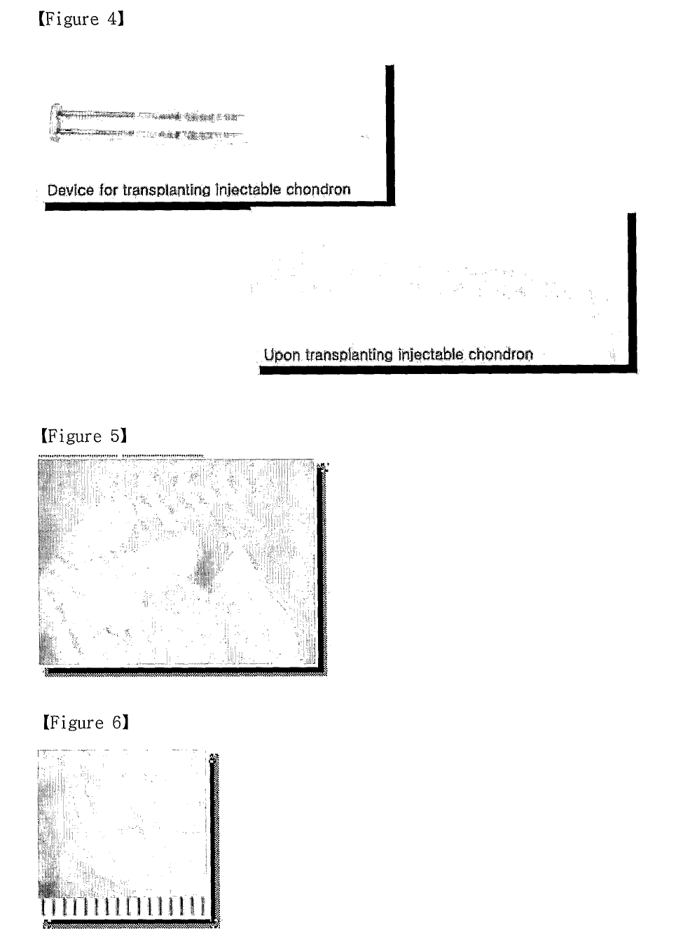

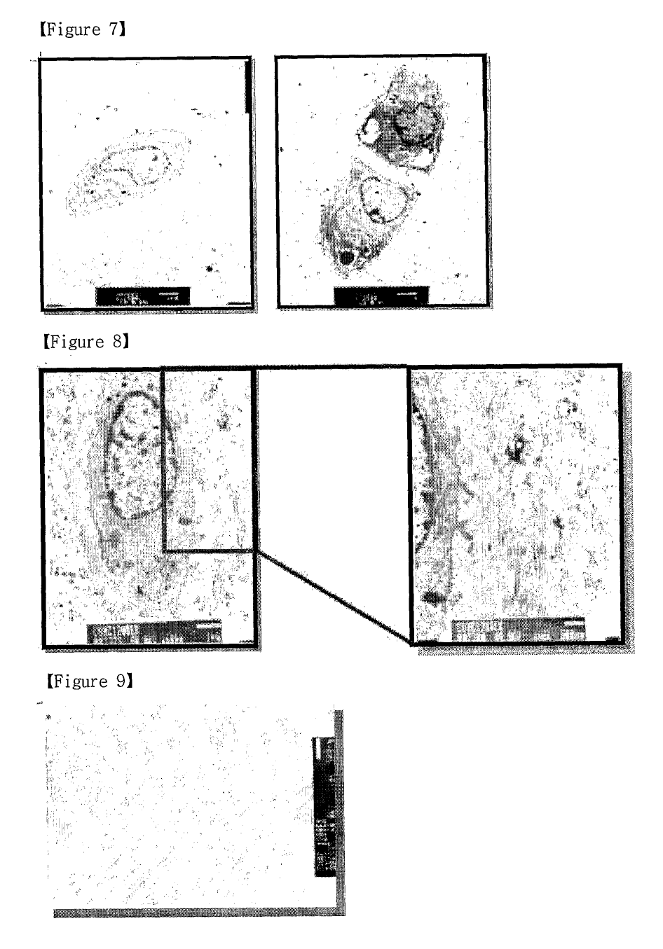

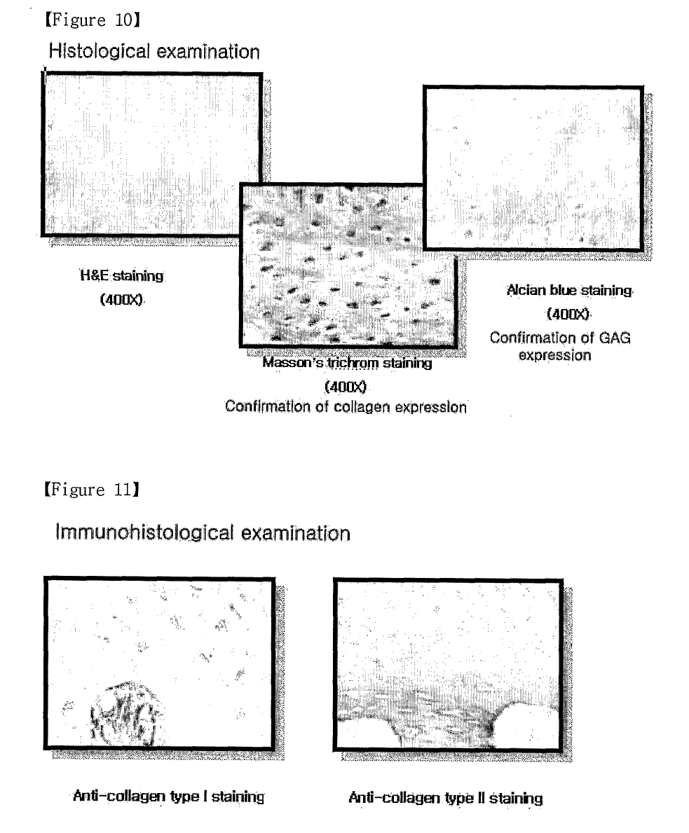

A method for transplantation of a cartilage therapeutic agent via injection, which is applied to the present invention, is constituted as shown in FIGS. 2 through 12.

In connection with the description of the present invention hereinafter, if it is considered that concrete description of known functions or constructions related to the present invention may make the subject of the present invention unclear, the detailed description thereof will be omitted.

Terms which will be described hereinafter are established taking into consideration the functions of the present invention and may vary according to the custom or manufacturer's intention. Therefore, the terms used herein should be defined based on the contents throughout the specification of the present invention.

The present invention is characterized by mixing matrices including fibrin, hyaluronic acid and collagen, which constitute the main ingredients of animal cartilage, via use of a syringe with a mixing tip, while injecting the resulting mixture into a damaged cartilage region.

That is, the present invention enables the following: convenient in situ transplantation due to rapid gelling characteristics in which an injectable cartilage therapeutic agent is gelated within about 1 to 5 minutes after transplanting; complete binding of transplanted sites to the adjacent native cartilage; treatment of a wide variety of cartilage defects and severe osteoarthritis; alleviation of the burden on patients during treatment via a simplified surgical operation method; and thereby promotion of faster and more effective generation of cartilage.

Further, the present invention provides a method for transplanting a cartilage therapeutic agent of the present invention via injection, according to the following example.

In this example, experiments using animal (canine) chondrocytes were carried out according to the following sequence. Experimental results were evaluated 3 months later. That is, as shown in FIG. 2:

1. The limbus of the cartilage defect region is trimmed and the size thereof is measured.
2. Among the contents in a kit, an aluminum cap of Vial No. 1 (Red) is removed and the cap and surface of the vial are cleaned with 70% ethanol. Vial No. 1 (Red) contains a cultured cell suspension of reddish color (chondrocyte culture+hyaluronic acid+aprotinin, etc.).
3. An aluminum cap of Vial No. 2 (Blue) is removed and the cap and surface of the vial are cleaned with 70% ethanol. Herein, Vial No. 2 (Blue) is a colorless vial containing a white or light yellow lyophilized powder (fibrinogen).
4. 1 ml of the content in Vial No. 1 (Red) is introduced into a 1 ml sterile syringe and the syringe needle is inserted into Vial No. 2 (Blue) which stands vertically. Herein, the contents of the syringe are directly injected over the lyophilizate, rather than injecting along the inner wall of the vial, while carefully preventing the needle tip from contacting the lyophilizate. Vial No. 2 (Blue) is then gently shaken by hand until the lyophilizate in Vial No. 2 (Blue) is completely dissolved.
5. An aluminum cap of Vial No. 3 (Red, small) is removed and the cap and surface of the vial are cleaned with 70% ethanol. Herein, Vial No. 3 (Red, small) is a colorless vial containing a white lyophilized powder (thrombin).
6. 1 ml of a solution contained in Vial No. 1 (Red) is introduced into a syringe and the syringe needle is inserted into Vial No. 3 (Red, small) which stands vertically, and the red liquid is gradually injected.
7. After removing an aluminum cap of Vial No. 4 (Yellow) and cleaning the cap and surface of the vial with 70% ethanol, 0.1 cc of the contents of Vial No. 3 (Red, small) is collected using a 1 ml syringe and is injected into Vial No. 4 (Yellow).
8. The entire contents of two vials containing chondrocyte suspension are introduced into Vial No. 4 (Yellow) using a 1 ml sterile syringe, followed by mixing two or three times using the syringe.
9. After confirming complete dissolution of the contents of Vial 2 (Blue), the entirety of the dissolved material is filled into a 1 ml syringe.
10. The well-mixed cell therapeutic of Vial 4 (Yellow) is introduced into a 1 ml syringe.
11. Two 1 ml syringes filled with the contents of Vial No. 2 (Blue) and Vial No. 4 (Yellow), respectively, are mounted on a standing syringe holder.
12. A screw connecting tip is mounted on the syringes, followed by a screw tip and a piston support.
13. After completion of the assembly, transplantation is initiated. Herein, if possible, injection of the cartilage therapeutic agent is carried out at once and without stopping.
14. About 5 minutes after completion of transplantation, the limbus of the transplantation site is trimmed.

A comparison between the present invention and conventional autologous chondrocyte transplantation (ACT) is shown in Table 1 below.

TABLE 1

| Conventional ACT | Inventive ACT (Injectable Chondron) |
|---|---|
| Liquid injectable preparation | Gel type (convenient transplantation due |
| | to rapid gelling characteristics within 1 |
| | to 5 minutes after transplanting, and |
| | readiness to form desired shapes) |
| Chondrocytes + culture medium | Chondrocytes + biocompatible materials |
| Osteoperiosteal grafting necessary | Periosteum unnecessary (transplantable |
| | via minimal incision) |
| Excellent cartilage regeneration ability | More advanced cartilage regeneration |
| | ability |
| Knee incision necessary (incision of | Capable of performing surgery via |
| more than 15 cm necessary for | minimal incision (5 cm), |
| periosteum collection and | Capable of performing surgery using an |
| transplantation, which requires suturing) | arthroscope |
| Uneven distribution (possibility of | Even distribution (exhibiting even |
| unequal and localized cartilage | distribution due to rapid gelling |
| regeneration by action of gravity, due to | characteristics within a period of 1 to 5 |
| suspending state of cells in a liquid | minutes after the moment of |
| phase) | transplantation and thereby |
| | homogeneous cartilage generation ability |
| | in that state) |
| Suture (the most complicated and time- | No suture |
| consuming process among surgical | |
| processes) | |
| Long Operation time | Short Operation time |
| Production of fibrocartilage by bleeding | Bleeding control with bone wax (mostly |
| | producing hyaline cartilage) |
| | Addition of column generating processes |
| | (see cartilage therapeutic agent |
| | composition and use thereof) |
| | Transplantable regardless of defect |
| | shapes |
| | Alleviation of cell leakage due to |
| | incomplete suture |
| | Reducing the time before resumption of |
| | weight-bearing activities due to no |
| | periosteum suture |

Meanwhile, as the medium for cell culture that can be used in the present invention, mention may be made of DMEM (Dulbecco's Modified Eagle's Medium), Ham's F-12, DMEM/F-12, IMDM (Iscove's Modified Dulbecco's Medium), McCoy's 5A Medium, MEM (Minimum Essential Medium), alpha-MEM, RPMI 1640, Leibovitz's L-15 Medium and Eagle's basal Medium, which are conventionally used in the art.

Preferably, the concentration of the lyophilized powder (fibrinogen) used herein is in the range of 10 mg/mL to 200 mg/mL. Where the concentration of fibrinogen is less than 10 mg/mL, it is too low to effectively act as the matrix. In contrast, where the concentration of fibrinogen is greater than 200 mg/mL, difficulty of dissolution occurs and it is difficult to use it due to very high viscosity. Therefore, it is preferred to use fibrinogen within the range of 10 mg/mL to 200 mg/mL.

In addition, the concentration of the white lyophilized powder (thrombin) used herein is preferably in the range of 1 IU/mL to 200 IU/mL. Where the concentration of thrombin is less than 1 IU/mL, it is too low to form fibrin, thereby being incapable of achieving gelation within several minutes. In contrast, where the concentration of thrombin is greater than 200 IU/mL, thrombin is solidified immediately upon mixing and thereby is not suitable for use in the injectable therapeutic agent. Therefore, it is preferred to use thrombin within the range of 1 IU/mL to 200 IU/mL.

Additionally, the concentration of aprotinin used herein is preferably in the range of 100 KIU/mL to 20,000 KIU/mL. Where the concentration of aprotinin is less than 100 KIU/mL, it is of little help to inhibit fibrin decomposition. In contrast, where the concentration of aprotinin is greater than 20,000 KIU/mL, cytotoxicity may occur. Therefore, it is preferred to use aprotinin within the range of 100 KIU/mL to 20,000 KIU/mL.

Further, the amount of hyaluronic acid used herein is preferably in the range of 0.01% to 2% (w/v). Where the amount of hyaluronic acid is less than 0.01%, it is difficult to sufficiently exert functions thereof as the matrix. In contrast, where the amount of hyaluronic acid is greater than 2%, bindability of fibrin may be structurally affected, thereby affecting the strength and hardness of the matrix. Therefore, it is preferred to use hyaluronic acid within the range of 0.01 to 2% (w/v).

Still further, the amount of collagen type 2 used herein is preferably in the range of 50 μg/mL to 1 mg/mL. Where the amount of collagen type 2 is less than 50 μg/mL, it is difficult to sufficiently exert functions thereof. In contrast, where the amount of collagen type 2 is greater than 1 mg/mL, characteristics thereof affect the bindability of fibrin, thereby affecting the strength and hardness of the matrix. Therefore, it is preferred to use collagen type 2 within the range of 50 μg/mL to 1 mg/mL.

As apparent from the foregoing, the present invention provides the following effects: alleviation of the inconvenience exhibited by conventional osteoperiosteal grafting, by mixing and transplanting matrices including fibrin, hyaluronic acid and collagen, which are the main ingredients of cartilage; in particular, convenient in situ transplantation due to rapid gelling characteristics in which an injectable cartilage therapeutic is gelated within a short period of time, i.e., about 1 to 5 minutes after transplanting; complete binding of transplanted sites to the adjacent native cartilage; treatment of a wide variety of cartilage defects and severe osteoarthritis; alleviation of the burden on patients during treatment via a simplified surgical operation method; and thereby promotion of faster and more effective cartilage generation, resulting in increased customer satisfaction.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1-4. (canceled)

5. A method of transplanting an injectable chondrocyte for autologous chondrocyte transplantation, comprising:
mixing matrices including fibrin, hyaluronic acid and collagen, which constitute the main ingredients of animal cartilage, via use of a syringe with a mixing tip, while injecting the resulting mixture into a damaged cartilage region,
placing 1 ml of a chondrocyte culture contained in Vial No. 1 (Red) into a 1 ml sterile syringe and injecting the cell culture into Vial No. 2 (Blue) containing white or light-yellow lyophilized powder (fibrinogen) by inserting the syringe into Vial No. 2 in the vertical direction, such that the concentration of fibrinogen is in the range of 10 mg/mL to 200 mg/mL;
injecting 1 ml of a cell culture contained in Vial No. 1 (Red) into Vial No. 3 (Red, small) containing white lyophilized powder (thrombin) by inserting the syringe into Vial No. 3 in the vertical direction, and gradually injecting red liquid;
collecting 0.1 cc of the contents of Vial No. 3 (Red, small) using a 1 ml syringe, followed by injection into Vial No. 4 (Yellow), such that the concentration of thrombin is in the range of 1 IU/mL to 200 IU/mL;
adding the entire contents of two vials containing chondrocyte suspension to Vial No. 4 (Yellow) using a 1 ml sterile syringe, followed by mixing the contents two or three times using the syringe;
confirming complete dissolution of the contents of Vial No. 2 (Blue) and then introducing the entirety of the dissolved material into a 1 ml syringe;
introducing the well-mixed cell therapeutic of Vial 4 (Yellow) into a 1 ml syringe; and
mounting two 1 ml syringes filled with the contents of Vial No. 2 (Blue) and Vial No. 4 (Yellow) on a standing holder followed by mounting a screw tip on the syringes, and mixing the contents of the syringes using the mixing tip thereof while injecting the resulting mixture into a damaged cartilage region of an animal.

6. The method according to claim 5, wherein the injecting step via insertion of a syringe needle is carried out by directly injecting the contents of the syringe over the lyophilizate, rather than injecting along the inner wall of the vial, while carefully preventing the needle tip from contacting the lyophilizate.

7. The method according to claim 5, wherein the cell culture contained in Vial No. 1 (Red) contains 0.01% to 2% (w/v) of hyaluronic acid, 100 to 20,000 KIU/mL of aprotinin, 50 μg/mL to 1 mg/mL of collagen type 2, or a mixture thereof.
